(19) United States
(12) Patent Application Publication
(10) Pub. No.: US 2008/0311158 A1
(43) Pub. Date: Dec. 18, 2008

(54) Hepatitis C Virus Replication System

(76) Inventor: Marcello Merola, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(21) Appl. No.: 11/664,007

(22) PCT Filed: Oct. 3, 2005
(86) PCT No.: PCT/IB05/03957

§ 371 Date: Feb. 29, 2008

(30) Foreign Application Priority Data
Oct. 1, 2004 (GB) 0421916.8
Jan. 25, 2005 (GB) 0501582.1

(51) Int. Cl. A61K 39/00; C12N 5/06; C12N 15/87; A61P 31/00; C12P 21/04

(52) U.S. Cl. 424/228.1; 435/366; 435/455; 435/69.3

(57) ABSTRACT

A cell including: (a) a hepatitis C virus genome, and/or nucleic acid encoding a hepatitis C virus genome; and (b) nucleic acid encoding one or both of hepatitis C virus proteins E1 and E2. The cells can be grown in culture in order to support HCV replication, and HCV virions can be purified from them.

All documents cited herein are incorporated by reference in their entirety.

### TECHNICAL FIELD

This invention is in the field of hepatitis C virus culture and protein expression.

### BACKGROUND ART

Due to the lack of an efficient in vitro culture system for hepatitis C virus (HCV), little is known about virus structure and assembly. A sub-genomic replicon containing most of the nonstructural proteins of HCV has been shown to replicate in a hepatoma cell line [1] and a further subgenomic replicon with improved transfection efficiency has also been disclosed [2,3]. These systems have allowed detailed studies on the HCV replication mechanism, but the absence of structural genes in the replicons means that virion assembly is not possible.

Reference 4 describes a method for persistent and transient replication of a full-length HCV genome in cell culture, in which mutant “selectable full-length” (sfl) genomes could stably replicate in 21-5 cells (derived from the Huh-7 cell line) and express all viral proteins. Although the mutations allowed RNA replication and a high efficiency of colony formation (ECF) when introduced into a selectable subgenomic replicon, the ECF was 3 to 4 orders of magnitude lower in the context of a sfl genome compared to the subgenomic replicons. Viral RNA was seen in the supernatant of the 21-5 cultures, although this effect was also seen with subgenomic replicons and so the authors believe that this release was by non-specific mechanisms, rather than by involvement of viral structural proteins.

Although in vitro assembly of HCV viral particles is possible in a cell-free system [5], efficient cell culture systems were not reported until 2005 [6-8]. The 21-5 cells described in reference 4 were found not to produce viral particles, and so there remains a need for an in vitro cell culture system that can support HCV full genome replication and that can produce viral particles, as well as further and improved systems compared to references 6 to 8.

### DISCLOSURE OF THE INVENTION

The authors of reference 4 suggested that the failure of 21-5 cells to support viral particle formation might be due to the lack of host cell factors. In contrast, the present inventors believe that the failure can be explained, at least in part, by the failure to express HCV structural envelope proteins E1 and E2. To overcome this deficiency, therefore, the inventors have chosen to complement these proteins in trans. Resulting cells are better able to support HCV replication and to produce HCV particles.

Thus the invention provides a cell including: (a) a hepatitis C virus genome, and/or nucleic acid encoding a hepatitis C virus genome; and (b) nucleic acid encoding one or both of hepatitis C virus proteins E1 and E2.

The invention also provides a cell in which: (a) a hepatitis C virus is replicating; and (b) hepatitis C virus protein(s) E1 and/or E2 is/are expressed in addition to any E1 and/or E2 protein that is expressed as a result of the hepatitis C virus life cycle. Thus E1 and/or E2 is/are expressed in a form separate from the E1/E2 that arises from proteolytic processing of HCV polyprotein expressed from the HCV genome during the viral life cycle. Two different types of RNA are produced: one type for the HCV RNA genome, and one for translation into E1 and/or E2 protein.

The invention also provides a cell that: (a) includes a hepatitis C virus genome, and/or nucleic acid encoding a hepatitis C virus genome; (b) expresses hepatitis C virus proteins E1 and E2; and (c) can grow in an in vitro culture.

The invention also provides a cell containing the following two nucleic acids in trans: (a) a hepatitis C virus genome; and (b) a nucleic acid encoding hepatitis C virus protein E1 and/or E2. The invention also provides a cell containing the following two nucleic acids in trans: (a) nucleic acid encoding a hepatitis C virus genome; and (b) a nucleic acid encoding hepatitis C virus protein E1 and/or E2.

The cells of the invention can be grown in culture in order to support HCV replication, and HCV virions and E1/E2 complexes can be purified from them.

The invention also provides a method for preparing a cell of the invention, comprising the steps of (a) introducing into the cell a hepatitis C virus genome and/or nucleic acid encoding a hepatitis C virus genome; (b) introducing into the cell nucleic acid encoding one or both of hepatitis C virus proteins E1 and E2. Steps (a) and (b) may be performed separately or simultaneously. The invention also provides an in vitro method for culturing hepatitis C virus in a cell, comprising said steps (a) and (b), followed by: (c) culturing the resulting cell.

The invention also provides a method for preparing hepatitis C virus E1 and E2 proteins, comprising the steps of: (a) culturing cells of the invention; and (b) purifying the E1 and E2 proteins from the cultured cells. The E1 and E2 proteins are preferably in the form of a complex, and the invention provides an E1/E2 complex obtainable by the method of the invention.

The invention also provides a cell including a hepatitis C virus genome, and/or nucleic acid encoding a hepatitis C virus genome, into which nucleic acid encoding one or both of hepatitis C virus proteins E1 and E2 can be introduced. Similarly, the invention provides a cell including nucleic acid encoding one or both of hepatitis C virus proteins E1 and E2, into which a hepatitis C virus genome and/or nucleic acid encoding a hepatitis C virus genome can be introduced. The invention also provides a vector comprising a sequence encoding the E1 and/or E2 proteins of HCV. This vector is for use in enhancing the ability of cells to support HCV replication in in vitro cell culture.

### The Cell

Cells of the invention can express the HCV genome, in order to support the HCV life cycle and its replication, and can also express E1 and/or E2 protein(s) separately from the E1 and E2 proteins that are produced during the HCV life cycle. Thus the production of E1 and E2 by proteolytic processing of viral polyprotein translated from the viral RNA genome is supplemented by protein(s) translated from a separate non-HCV RNA (e.g. from a cellular mRNA).

As the E1 and E2 proteins of HCV are naturally glycosylated, the invention will generally use a eukaryotic cell. It is preferred to use a mammalian cell, such as a primate cell, including a human cell. As HCV naturally infects liver cells then it is convenient to use a cell derived from liver.

Typically, the cells used in the invention will be cell lines, and preferably packaging cell lines. A preferred cell line for use with the invention is derived from a hepatocellular carcinoma, namely the human hepatoma cell line known as ‘Huh7’ [9]. The most preferred cell line is the Huh7-derived cell line known as ‘21-5’, which supports a full length HCV replicon. Other preferred cell lines are Huh-7.5, Huh-7.5.1 [8] and Huh-7.8, which are sub-lines of Huh-7 that can support complete replication in cell culture. Cells derived by passaging of Huh7 cells (and their derivatives) can also be used, as well as cells derived by treating Huh7 cells with α-interferon and/or γ-interferon. As disclosed in reference [10], cell lines permissive for HCV can be prepared by a process comprising (a) culturing cells infected with HCV; (b) curing the cells of HCV; and (c) identifying a sub-line of the cured cells that is permissive for HCV replication.

Other established human hepatoma and hepatoblastoma cell lines include HuH-6 cl-5, PLC/PRF/5, huH-1, and huH-4. Huh7 cells can be grown as monolayers in media such complete DMEM (e.g. Dulbecco's modified minimal essential medium supplemented with 2 mM L-glutamine, nonessential amino acids, 100 U/ml penicillin, 100 μg/ml streptomycin, 10% fetal calf serum).

Particular cell types may be preferred for use with particular viral strains, and vice versa.

Cells of the invention express polyprotein from the HCV genome, and also express supplemental E1 and/or E2 protein(s). As a result, the cells include the protein building blocks for expressing HCV particles, including virions and virus-like particles (VLPs). Virions and VLPs can thus be prepared from the cells of the invention. These may or may not include a RNA genome. The cells can also express complexes of E1 and E2. The cells may also express complexes of E1, E2, NS3 and NS5a.

As described below in more detail, cells of the invention include a HCV genome and/or nucleic acid encoding a HCV genome. A HCV genome can be introduced into a cell by viral infection, or can be produced in situ either after viral infection (real or artificial) or after transcription from a DNA copy of the genome. Methods for introducing HCV genomes or DNA encoding a HCV genome into a cell are routine in the art. HCV genomes that have been transcribed in vitro can be introduced into Huh7 cells using electroporation, as disclosed in reference 4 (4×10⁶ Huh-7 cells electroporated with 1 μg of purified in vitro transcripts, or 2.4×10⁷ Huh-7 cells electroporated with 60 μg of HCV RNA).

Cells of the invention also include nucleic acid that encodes E1 and/or E2 protein. Methods for introducing such nucleic acids are routine in the art.

When the HCV genome and the E1/E2 nucleic acid are both expressed, dot-like particles can be seen if immunofluorescent straining is used. If desired, release of these particles from the cells can be facilitated by treating them with suitable release agents e.g. to cause the release of exosomes, etc. Such agents include alcohol, stress conditions, etc. Non-covalent E1/E2 heterodimers have been detected in the endoplasmic reticulum, and can be recovered from this cellular compartment.

### The Hepatitis C Virus Genome

Cells of the invention include a HCV genome and/or nucleic acid encoding a HCV genome. Thus the cells may include a + strand single-stranded RNA genome, or they may include nucleic acid that can be transcribed to give, either directly or indirectly, a + strand RNA genome. For instance, transcription of a DNA copy of the genome in the correct orientation (even though the natural HCV life cycle does not include a DNA stage) gives a RNA that can act as a HCV genome i.e. it can direct expression of the HCV polyprotein, which is then processed as seen in the normal HCV life cycle.

The HCV genomic RNA naturally includes a 5′ cap (m⁷G5′ppp5′A) and no poly-A tail, with both 5′ and 3′ noncoding regions (NCRs). These elements will typically be present in HCV genomes used according to the invention, but the HCV genome and/or the nucleic acid encoding the genome may include one or more elements not seen in native genomes. For instance, it is normal for HCV genomes used in in vitro replication systems to include one or more of the following: a sequence encoding a selection marker (such as a neomycin resistance marker) e.g. near the 5′ end of the genome, upstream of the polyprotein; an internal ribosome entry site (IRES) such as from EMCV e.g. upstream of the polyprotein coding sequence, to permit translation of the polyprotein even though there may be other upstream coding sequences. A construct with a 5′ T7 promoter at the 5′ end of the genome has been described [101], but this is not preferred.

The HCV genome can be of any type (e.g. 1, 2, 3, 4, 5, 6) or subtype (1a, 1b, 1c, 1d, 1e, 1f, 1g, 1h, 1i, 1j, 1k, 1l, 1m, 2a, 2b, 2c, 2d, 2e, 2f, 2g, 2h, 2i, 2k, 2l, 2m, 3a, 3b, 3c, 3d, 3e, 3f, 3g, 3h, 3i, 3k, 4a, 4c, 4d, 4e, 4f, 4g, 4h, 4k, 4l, 4m, 4n, 4o, 4p, 4q, 4r, 4s, 4t, 5a, 6a, 6b, 6d, 6f, 6g, 6h, 6i, 6j, 6k, 6l, 6m, 6n). This nomenclature is the current standard, as set out by the NIAID Hepatitis C Virus (HCV) Sequence Database [11] in which previous genotypes 7-9 have been reclassified as subtypes of type 6, based on reference 12. Thus the new classification includes previous classifications I, II, III, IV, V, VI, 4a, 4p, 7a, 7b, 7c/NGII/VII, 7d, NGI, 8a, 8b, 9a, 9b, 9c, 10a/TD3 and 11a. One suitable HCV strain for use with the invention is the genotype 2a strain JFH1 [6]. The HCV genome may include one or more mutations (including insertions, deletions and/or substitutions) relative to a wild-type genome, or may be a hybrid of more than one wild-type genome e.g. a chimera of a subtype 2a strain (e.g. J6) and a subtype 1a strain (e.g. H77) [7] Mutations in the genome are frequent, as the viral RNA replicase lacks proof-reading activity, and resulting mutant genomes (or ‘quasi-species’) can readily be obtained and analysed. Reference 4 describes mutants for cell-adaptation, including E1202G, T12801, K1846T and S2197P. Reference 3 describes HCV variants that include mutations (e.g. R1164G, S1172C, S1172P, A1174S, S11791) that increase transfection efficiency and the ability to survive subpassages. Other known mutations include, but are not limited to, L17571, N2109D, P2327S and K2350E. Particular mutations may be preferred for use with particular host cells and vice versa. Although the polyprotein of the HCV preferably includes all of C, E1, E2, p7, NS2, NS3, NS4A, NS4B, NS5A and NS5B, larger deletions and insertions are also possible (e.g. deletion of mature coding regions, such as of non-structural proteins, or insertion of non-HCV sequences). The invention can also be used with subgenomic HCVs (i.e. including less than a full genome, typically including non-structural proteins but not structural proteins e.g. lacking full C, E1, E2), but a key advantage of the invention is that it permits the replication of full-length genomes.

These and other mutations can be present in the HCV genome used according to the invention, but these mutants will generally not remove the ability of the genome to direct expression of a HCV polyprotein that can then replicate the HCV genome from which it was expressed.

The HCV RNA or the nucleic acid encoding the HCV RNA may be introduced into a cell, or may already be part of the cell e.g. the 21-5 cell line [4] is already infected with HCV.

Where the cell includes nucleic acid that encodes a HCV genome, this can take the form of DNA or RNA, and can either be chromosomal or extra-chromosomal (e.g. episomal). A DNA plasmid that encodes the HCV genome can conveniently be used.

DNA encoding both the HCV genome and the E1/E2 proteins can be used. This DNA will usually have two separate transcriptions: (i) the HCV genome (or possibly the anti-genome), and (ii) mRNA encoding the E1/E2 proteins, but it is also possible to have a single transcript including: (i) the HCV genome (or anti-genome) and coding sequences for E1/E2, with an IRES to control translation of the downstream sequence.

A HCV genome will typically be included in the form of a replicon i.e. a nucleic acid that is capable of directing the generation of copies of itself. Cell-based HCV replication systems that use a genomic or subgenomic replicon system are well known. Replicons are based on the sense strand of the viral RNA, but the invention can also utilise a complementary sequence that can be converted into the sense strand to provide a replicon. Replicons may also contain non-HCV genes, e.g. reporter genes.

Nucleic Acid Encoding E1 and/or E2

Cells of the invention include nucleic acid that encodes one or, preferably, both of E1 and E2. This nucleic acid is separate from the HCV genome and from any nucleic acid that encodes the HCV genome. The sequences of supplemental E1 and E2 will preferably, however, be the same as the E1 and E2 sequences found in the HCV polyprotein.

The nucleic acid encoding E1 and/or E2 may be DNA or RNA. DNA vectors are preferred. The nucleic acid can either be chromosomal or extra-chromosomal (e.g. episomal).

A preferred nucleic acid vector for introducing E1 and E2 to a cell is a retroviral vector, which may be an integrating vector. The E1 and E2 sequences may therefore be introduced as inserts in the RNA genome of a retrovirus. After reverse transcription (and, where applicable, integration) the E1 and E2 sequences will be in DNA form within the cell. Other suitable vectors include DNA plasmids.

Where both E1 and E2 are expressed, these may be expressed from the same nucleic acid or may be expressed from different nucleic acids. For example, one plasmid encoding both proteins could be used (e.g. two different genes, or one gene with an IRES) or two plasmids could be used. Moreover, the proteins may be translated separately from each other or may be translated as a single polypeptide that is proteolytically cleaved in the same way as the HCV polyprotein to give separate E1 and E2.

Control of E1/E2 expression will generally be under the control of a promoter. The invention may use constitutive promoters or may use controllable promoters. Preferred promoters are from glycolytic enzymes, such as the phosphoglycerate kinase (PGK) promoter. Human promoters are preferred, and preferably HCV E1 and/or E2 will be under the control of the human phosphoglycerate kinase promoter (hPGK).

In addition to expressing E1 and E2, the nucleic acid(s) may express p7. As an alternative, p7 may be expressed from the same nucleic acid as only one of E1 or E2, or may be expressed from a different nucleic acid from E1 and E2. For example, one plasmid each for E1, E2 and p7 could be used.

### E1/E2 Compositions

E1 and E2 proteins have been found to co-localise when expressed according to the invention, and this permits their co-purification from cells. The E1/E2 complexes (e.g. heterodimers) can then be used e.g. as the active ingredient in an immunogenic composition, such as a vaccine for treating and/or preventing HCV infection.

Virions and/or VLPs produced by cells of the invention can also be used as the active ingredient in immunogenic compositions. If RNA is included in these virions/VLPs then the RNA is preferably sub-genomic.

Compositions of the invention may be pharmaceutical compositions that include a pharmaceutically acceptable carrier. Such compositions can be prepared using a process comprising the step of admixing E1/E2 complexes with the pharmaceutically acceptable carrier.

Typical ‘pharmaceutically acceptable carriers’ include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, sucrose, and the like, may be present. Sterile pyrogen-free, phosphate-buffered physiologic saline is a typical carrier (e.g. based on water for injection). A thorough discussion of pharmaceutically acceptable excipients is available in reference 13.

Compositions of the invention will typically be in aqueous form (e.g. solutions or suspensions) rather than in a dried form (e.g. lyophilised). Aqueous compositions are also suitable for reconstituting other materials from a lyophilised form. Where a composition of the invention is to be used for such extemporaneous reconstitution, the invention also provides a kit, which may comprise two vials, or may comprise one ready-filled syringe and one vial, with the aqueous contents of the syringe being used to reactivate the dried contents of the vial prior to injection.

Compositions of the invention may be presented in vials, or they may be presented in ready-filled syringes. The syringes may be supplied with or without needles. Compositions may be packaged in unit dose form or in multiple dose form. A syringe will generally include a single dose of the composition, whereas a vial may include a single dose or multiple doses. For multiple dose forms, therefore, vials are preferred to pre-filled syringes.

Effective dosage volumes can be routinely established, but a typical human dose of the composition has a volume of about 0.5 ml e.g. for intramuscular injection.

The pH of the composition is preferably between 6 and 8, and more preferably between 6.5 and 7.5 (e.g. about 7). Stable pH may be maintained by the use of a buffer e.g. a Tris buffer, a phosphate buffer, or a histidine buffer. Compositions of the invention will generally include a buffer. If a composition comprises an aluminium hydroxide salt, it is preferred to use a histidine buffer [14] e.g. at between 1-10 mM, preferably about 5 mM. The composition may be sterile and/or pyrogen-free. Compositions of the invention may be isotonic with respect to humans. Compositions are preferably free from HCV RNA and/or from HCV virions.

Compositions of the invention are immunogenic, and are more preferably vaccine compositions. Vaccines according to the invention may either be prophylactic (i.e. to prevent infection) or therapeutic (i.e. to treat infection), but will typically be prophylactic. Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Compositions may be prepared as injectables, either as liquid solutions or suspensions. The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 15 & 16]. Injectables for intramuscular administration are typical.

Compositions of the invention may include an antimicrobial, particularly when packaged in multiple dose format. Antimicrobials such as thiomersal and 2-phenoxyethanol are commonly found in vaccines, but it is preferred to use either a mercury-free preservative or no preservative at all.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity. A concentration of 10±2 mg/ml NaCl is typical. The concentration of sodium chloride is preferably about 9 mg/ml.

Compositions of the invention will generally be administered in conjunction with other immunoregulatory agents. In particular, compositions will usually include one or more adjuvants, and the invention provides a process for preparing a composition of the invention, comprising the step of admixing vesicles of the invention with an adjuvant e.g. in a pharmaceutically acceptable carrier. Suitable adjuvants include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 17], or mixtures of different mineral compounds, with the compounds taking any suitable form (e.g. gel, crystalline, amorphous, etc.), and with adsorption being preferred. The mineral containing compositions may also be formulated as a particle of metal salt [18].

A typical aluminium phosphate adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose. Where an aluminium phosphate it used and it is desired not to adsorb an antigen to the adjuvant, this is favoured by including free phosphate ions in solution (e.g. by the use of a phosphate buffer).

A typical dose of aluminium adjuvant is about 3.3 mg/ml (expressed as Al³⁺ concentration).

### B. Oil Emulsions

Oil emulsion compositions suitable for use as adjuvants in the invention include squalene-water emulsions, such as MF59 [Chapter 10 of ref. 17; see also ref. 19] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

### C. Saponin Formulations [Chapter 22 of Ref. 17]

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 20. Saponin formulations may also comprise a sterol, such as cholesterol [21].

Combinations of saponins and cholesterols can be used to form unique particles called immunostimulating complexs (ISCOMs) [chapter 23 of ref. 17]. ISCOMs typically also include a phospholipid such as phosphatidylethanolamine or phosphatidylcholine. Any known saponin can be used in ISCOMs. Preferably, the ISCOM includes one or more of QuilA, QHA and QHC. ISCOMs are further described in refs. 21-23. Optionally, the ISCOMs may be devoid of extra detergent [24].

A review of the development of saponin based adjuvants can be found in refs. 25 & 26.

D. Virosomes and virus-like particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Q13-phage (such as coat proteins), GA-phage, fr-phage, AP205 phage, and Ty (such as retrotransposon Ty protein pl). VLPs are discussed further in refs. 27-32. Virosomes are discussed further in, for example, ref. 33

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 34. Such “small particles” of 3dMPL are small enough to be sterile filtered through a 0.22 μm membrane [34]. Other non-toxic LPS derivatives include monophosphoryl lipid A mimics, such as aminoalkyl glucosaminide phosphate derivatives e.g. RC-529 [35,36].

Lipid A derivatives include derivatives of lipid A from Escherichia coli such as OM-174. OM-174 is described for example in refs. 37 & 38.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 39, 40 and 41 disclose possible analog substitutions e.g. replacement of guanosine with 2′-deoxy-7-deazaguanosine. The adjuvant effect of CpG oligonucleotides is further discussed in refs. 42-47.

The CpG sequence may be directed to TLR9, such as the motif GTCGTT or TTCGTT [48]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 49-51. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 48 & 52-54.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 55 and as parenteral adjuvants in ref. 56. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 57-64. Numerical reference for amino acid substitutions is preferably based on the alignments of the A and B subunits of ADP-ribosylating toxins set forth in ref. 65, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [66], etc.) [67], interferons (e.g. interferon-γ), macrophage colony stimulating factor, and tumor necrosis factor.

### G. Bioadhesives and Mucoadhesives

Bioadhesives and mucoadhesives may also be used as adjuvants in the invention. Suitable bioadhesives include esterified hyaluronic acid microspheres [68] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [69].

### H. Microparticles

Microparticles may also be used as adjuvants in the invention. Microparticles (i.e. a particle of ˜100 nm to ˜150 μm in diameter, more preferably 200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of Ref. 17)

Examples of liposome formulations suitable for use as adjuvants are described in refs. 70-72.

### J. Polyoxyethylene Ether and Polyoxyethylene Ester Formulations

Adjuvants suitable for use in the invention include polyoxyethylene ethers and polyoxyethylene esters [73]. Such formulations further include polyoxyethylene sorbitan ester surfactants in combination with an octoxynol [74] as well as polyoxyethylene alkyl ethers or ester surfactants in combination with at least one additional non-ionic surfactant such as an octoxynol [75]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Polyphosphazene (PCPP)

PCPP formulations are described, for example, in refs. 76 and 77.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e.g. “Resiquimod 3M”), described further in refs. 78 and 79.

The invention may also comprise combinations of aspects of one or more of the adjuvants identified above. For example, the following adjuvant compositions may be used in the invention: (1) a saponin and an oil-in-water emulsion [80]; (2) a saponin (e.g. QS21)+a non-toxic LPS derivative (e.g. 3dMPL) [81]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [82]; (5) combinations of 3dMPL with, for example, QS21 and/or oil-in-water emulsions [83]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 17.

The use of aluminium salt adjuvants is particularly preferred, and antigens are generally adsorbed to such salts. It is possible in compositions of the invention to adsorb some antigens to an aluminium hydroxide but to have other antigens in association with an aluminium phosphate. In general, however, it is preferred to use only a single salt e.g. a hydroxide or a phosphate, but not both. Not all vesicles need to be adsorbed i.e. some or all can be free in solution.

### Methods of Treatment

The invention also provides a method for raising an immune response in a mammal, comprising administering a pharmaceutical composition of the invention to the mammal. The immune response is preferably protective and preferably involves antibodies. The method may raise a booster response in a patient that has already been primed against HCV.

The mammal is preferably a human. Where the vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc.

The invention also provides E1/E2 complexes of the invention for use as a medicament. The medicament is preferably able to raise an immune response in a mammal (i.e. it is an immunogenic composition) and is more preferably a vaccine.

The invention also provides the use of E1/E2 complexes of the invention in the manufacture of a medicament for raising an immune response in a mammal.

These uses and methods are preferably for the prevention and/or treatment of a disease caused by HCV e.g. hepatitis.

Methods for checking efficacy of therapeutic hepatitis treatment are known in the art. One way of checking efficacy of prophylactic treatment involves monitoring immune responses against E1/E2 antigens after administration of the composition. Immunogenicity of compositions of the invention can be determined by administering them to test subjects (e.g. children 12-16 months age, or animal models) and then determining standard parameters such as ELISA titres (GMT). These immune responses will generally be determined around 4 weeks after administration of the composition, and compared to values determined before administration of the composition.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

The invention may be used to elicit systemic and/or mucosal immunity.

### General

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

Detailed information about hepatitis C virus, including its life cycle, replication, culture conditions, genome, polyprotein, proteolytic processing, etc., can be found in chapters 32 to 34 of reference 84.

### BRIEF DESCRIPTION OF DRAWINGS

FIG. 1 shows a schematic diagram of the method used to generate and infect cells with the rvE1809 (R809) retroviral vector.

FIG. 2 shows a western blot of HepG2, Huh-7 or 21-5 cells transfected with the R809 vector. The staining antibody is anti-E1/E2.

FIGS. 3 and 4 show western blots of 21-5 cells (and, in FIG. 3, the culture supernatant) stained with anti-E1/E2 antibody after immunoprecipitation using anti-E1E2, anti-core or anti-E2.

FIG. 3 is a non-reducing gel, whereas

FIG. 4 is a reducing gel.

FIG. 5 is similar to FIG. 3, but the staining antibody is anti-core rather than anti-E1/E2.

FIGS. 6 and 7 show intracellular immunofluorescence analysis of 21-5/R809 cells. Cells were stained with either anti E1/E2 or anti core. An overlay of the two individual stains indicates a co-localisation of E1/E2 and core.

FIG. 8 shows an immunofluoresence analysis of individual 21-5/R809 cells. Some cells expressing E1/E2 were not expressing core protein and some cells expressing core were not expressing E1/E2.

FIG. 9 shows an intracellular immunofluoresence analysis of 21-5/R809 cells. E1 and E2 are structured in ‘dots’ with variable shape, dimension and individual distribution.

FIG. 10 shows an intracellular immunofluoresence analysis of 21-5/R809 cells. ‘Dots’ of E1 and E2 show partial co-localisation with the non-structural protein NS3.

FIG. 11 shows intracellular immunofluorescence analysis of Huh7/R809 lacking the HCV replicon. Cells were stained with anti E1/E2. E1/E2 show a dotted distribution but labelling was around 5-fold lower than in 21-5/R809 cells.

FIG. 12 shows a 3-dimensional graphical representation of the distribution of E1/E2 along cells with a maximum length of about 3 μm.

FIGS. 13 and 14 show intracellular immunofluoresence analysis of 21-5/R809 cells. Cells were stained with anti E1/E2 and either anti calnexin (CNX), or anti ERP60. Overlays suggest that E1/E2 are not co-localising completely with the standard ER markers CNX and ERP60.

FIG. 15 shows intracellular immunofluoresence analysis of 21-5/R809 cells. Cells were stained with anti E1/E2 and anti GM130. The overlay suggests that E1/E2 and GM130 do not co-localise.

FIG. 16 shows intracellular immunofluoresence analysis of 21-5/R809 cells before and after treatment of the cell cultures with IFN-α. Cells were stained with anti E1/E2. Cells treated with IFN-α lose the typical ER-pattern distribution of E1 and E2.

FIGS. 17 and 18 show levels of HCV RNA or ribosomal RNA after IFN-α treatment of cell culture.

FIG. 19 shows a western blot of fractions after differential centrifugation.

FIG. 20 shows results of a membrane flotation analysis demonstrating that the majority of E1 and E2 associate with NP-40 resistant membranes and co-fractionate with caveolin-2, a marker for intracellular lipid raft.

FIG. 21 shows the expression of the HCV glycoproteins E1/E2 in replicon cell lines. (A) VSV-pseudotyped particles were produced in HEK293T cells cotransfected with plasmids encoding the HIV gag/pol proteins, the viral envelope protein (VSV-G), the HIV regulatory protein rev, and the self inactivating lentiviral vector encoding the encapsidation signal and the E1E2p7 transcriptional unit under the control of the human phoshoglycerokinase (hPGK) promoter. (B) E1/E2 expression in Huh-7 and 21-5 transduced cell lines was detected by immunofluorescence analysis using the anti-E1/E2 chimpanzee antisera L559 [85]. E1/E2 are visualized with red color, while blue represents DAPI staining for nuclei. (C) Cell lysates from 21-5 and 21-5 R809 were immunoprecipitated with the conformational monoclonal antibody CBH-2 and the immunoprecipitates were blotted with the anti-E1/E2 chimpanzee antisera L559 to reveal both E1 and E2.

FIG. 22 shows HCV glycoproteins E1 and E2 colocalize with core, NS3 and NS5A. One day after seeding, 21-5 and 21-5R809 were double stained with a mouse monoclonal antibody against core, NS3 or NS5A (originally detected as a green colour) and the anti-E1/E2 chimpanzee antisera L559 (originally detected as a red colour).

FIG. 23 shows the colocalization of HCV structural and nonstructural proteins with the nascent RNA. (A) One day after seeding, 21-5R809 cells were fixed and stained with the anti-E1/E2 chimpanzee antisera L559 (originally red), the mouse monoclonal antibody 7-D4 against NS5A (originally green) and the mouse monoclonal antibody MMM33 against NS3 (originally blue). (B) 21-5R809 were transfected using lipofectamine 2000 and labeled with BrUTP for 2 hours or 4 hours. The cells were stained with the anti-E1/E2 chimpanzee antisera L559 (originally red), the mouse monoclonal antibody MMM33 against NS3 (originally blue) and the monoclonal antibody against BrdUTP (originally green). In (C) cells were pulsed for 4 hours and stained as in (B). Bottom and lateral strips show z-sections through the indicated lines, showing co-localization of E1/E2, NS3 and RNA in the speckle like structures. Bars, 5 μm.

FIG. 24 shows the distribution of HCV nonstructural proteins in different cell lines supporting HCV RNA replication. One day after seeding, 21-5 harboring full length replicon, NS3-3′ and NS3-3′R809 harboring subgenomic replicon were fixed and stained with the mouse monoclonal antibody 7-D4 against NS5A (originally red) and the mouse monoclonal antibody MMM33 against NS3 (originally green). In the bottom panels, insets show E1/E2 staining with the anti-E1/E2 chimpanzee antisera L559 (originally blue). Bars, 5 μm.

FIG. 25 shows the intracellular localization of the HCV glycoproteins E1 and E2. (A) 21-5R809 were fixed and stained with the indicated antibodies. Only merged images are shown. (B) Magnification on 21-5R809 cells stained with the anti-E1/E2 chimpanzee antisera L559 (originally red), a rabbit polyclonal antibody against calnexin (originally green) and the mouse monoclonal antibody MMM33 against NS3 (originally red, middle panel or green, lower panel). Bars, 2.2 μm.

FIG. 26 shows the subcellular fractionation and detergent solubilization of HCV proteins. (A) Cell lysates from 21-5R809 cells were untreated, treated with 1% TX-100 on ice or 1% TX-100 at 37° C. Subcellular fractionation was done by discontinuous sucrose gradient centrifugation. Fractions were collected from the top to the bottom of the gradient. Equal volumes of the recovered fractions were analyzed on a 10% SDS-PAGE, transferred to nitrocellulose membranes and immunoblotted with antibodies against E1/E2, NS3, core, Caveolin-2, calnexin. Fractions are numbered from 1 to 11, from top to bottom. (B) Density profile of the collected fractions.

### MODES FOR CARRYING OUT THE INVENTION

### Reinforcing Glycoprotein Expression in the HCV Genomic Replicon System

To define the nature and the subcellular compartmentalization of the HCV replication/assembly complex, we studied the localization of HCV structural and nonstructural proteins in 21-5 cells harboring a full-length genotype 1b replicon [4]. In these cells persistent RNA replication was verified by Northern blot and protein expression by Western Blot and ³⁵S-Met/Cys labeling followed by immunoprecipitation. Using these techniques, we revealed the presence of core, NS3, NS5a and NS5b in cellular extracts. Unexpectedly, the expression of E1 and E2 was undetectable by Western blot, metabolic labeling or immunofluorescence analysis.

To determine if the low level of the glycoprotein expression caused the inability of the system to produce viral particles [4], we decided to provide the 21-5 cells with stable expression of E1 and E2 using gene transfer by lentiviral vectors [86]. We cloned the E1E2p7 coding region of HCV 1a genotype into a self-inactivating lentiviral vector downstream of the ubiquitously expressed phosphoglycerate kinase promoter (PGKp) [87] and generated VSV-pseudotyped particles

### Cell Lines

Three different cell lines were used in this study: 1) naïve Huh7; 2) Huh-21-5, with the full length replicon I₃₈₉neo/core-3′/5.1 [4], Huh-5-15, with the subgenomic replicon I₃₈₉neo/NS3-3′ [1]. These cells were grown in complete DMEM plus G418 (Geneticin; Life Technologies) for cell lines carrying HCV replicons (250 μg/ml for Huh-21-5; 750 μg/ml for Huh-5-15). Huh7 and 21-5 cells [4] were obtained from a commercial source. HepG2 cells were also used for comparison. Huh7 cells are not infected with HCV, but 21-5 cells are. Lentiviral vector production and transduction of the HCV replicon cell lines.

To generate the PGK/HCV E1E2p7 lentiviral vector, we amplified a 1935 base-pair fragment coding from Tyr₁₆₄ to Ala₈₀₉ of HCV 1a genotype using sense and antisense primers in which a Met residue was added upstream the Tyr₁₆₄ and a stop codon downstream Ala₈₀₉. We replaced the green fluorescence protein (GFP) of the self-inactivating lentiviral vector pCCLsin.PPT.hPGK.GFP.Wpre [88], [87] with the amplified E1E2p7 fragment.

Retroviral particles containing a RNA encoding E1 and E2 proteins were prepared by the process shown in FIG. 1. DNA encoding the E1, E2 and p7 proteins, flanked by viral LTRs (10 μg of pCCL.sin.PPT.hPGK.EIE2p7), was transiently transfected into HEK293T cells plated on 10 cm dishes by calcium-phosphate, along with (i) DNA encoding VSV-G (vesicular stomatitis virus glycoprotein) under the control of a cytomegalovirus (CMV) promoter (3.5 μg), (ii) DNA encoding REV under the control of a RSV promoter (2.5 μg), and (iii) DNA encoding GAG and POL under the control of a CMV promoter. The transfected cells produced retroviral particles containing E1E2 RNA, and these particles could be collected from the supernatant of a culture of the HEK293T cells. Two collections of viral supernatant were made 24 and 48 hours after transfection. After filtering through a 0.2-μm filter (Millipore, Bedford, Mass.), the lentiviral vector was concentrated by ultracentrifugation. The purified particles (either 50 μl or 100 μl) were used to infect naïve Huh-7, Huh-5-15 or 21-5 cells by 6-hour exposure to the concentrated lentiviral vector in the presence of polybrene (8 μg/ml) at 37° C. and 5% CO₂, and these cells were analysed 96 hours after transduction for expression of E1 and E2 proteins by flow cytometry and Western blotting. The retroviral vector was named ‘rvE1809’ or ‘R809’.

Lentiviral transduction of both naïve Huh-7 and 21-5 cells worked very efficiently, allowing stable expression of HCV E1 and E2 in almost 100% of these cells, as assessed by FACS analysis on permeabilized cells. We refer to the cells transduced with HCV-1a E1E2p7 as R809.

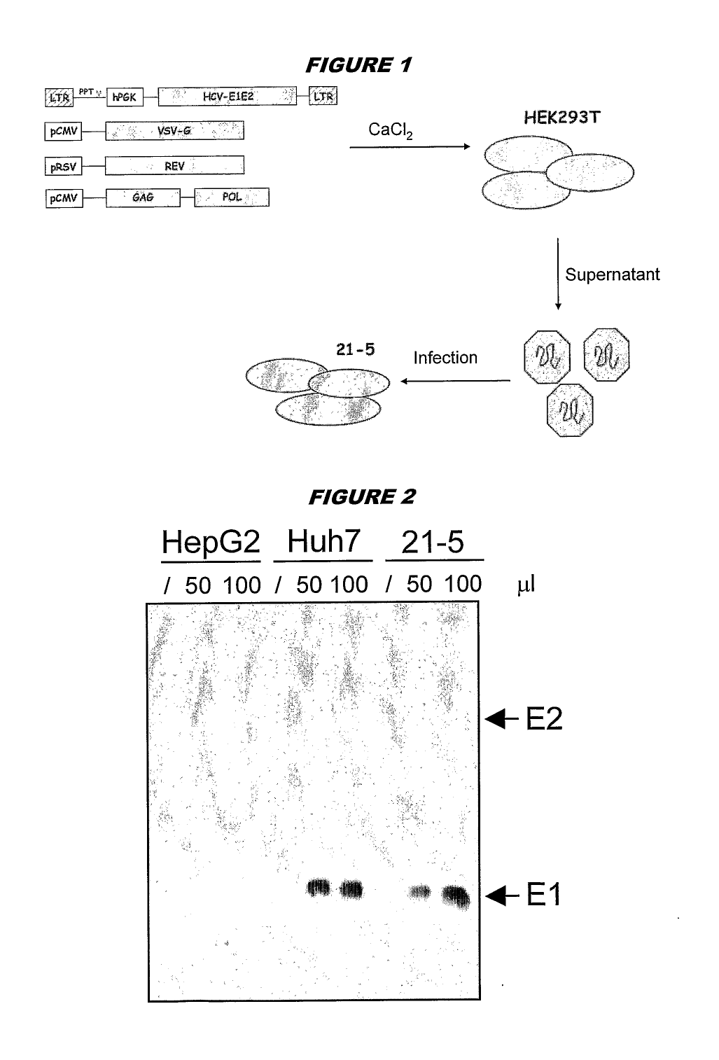

Anti-E1/E2 antibody was used in western blots, and E1 and E2 were seen in both Huh7 and 21-5 cells (FIG. 2). This analysis also confirmed that E1 and E2 are processed by the necessary signal peptidases.

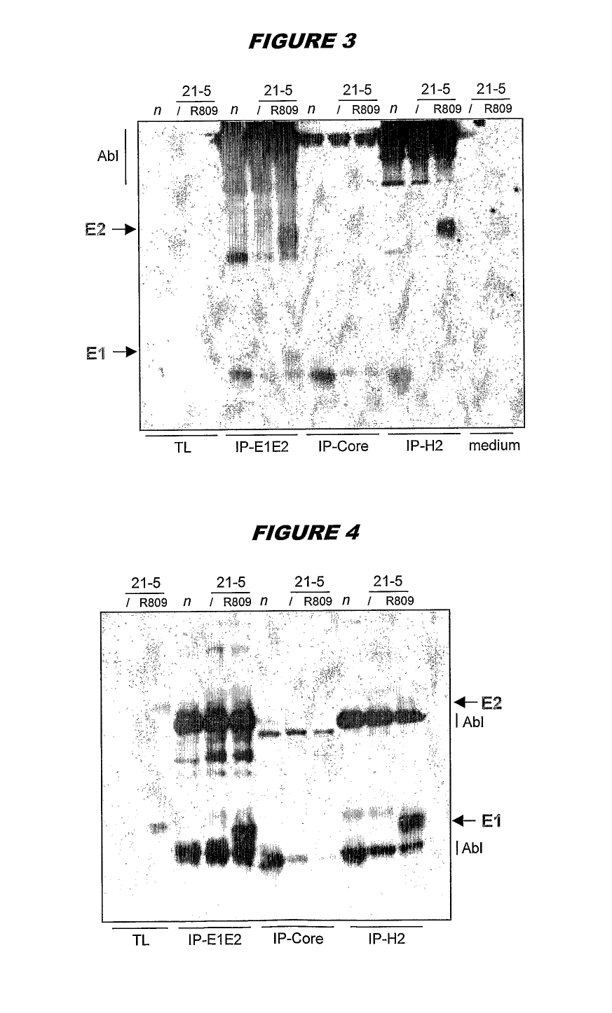

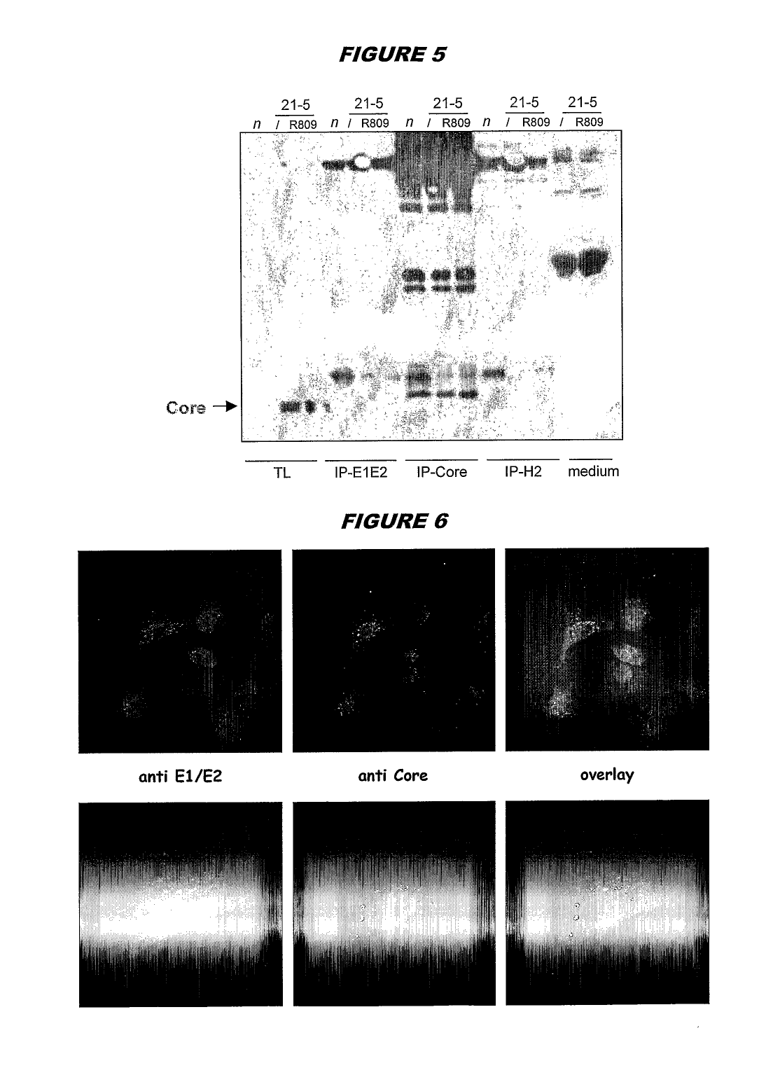

Immunoprecipitation experiments were performed on 21-5 cells using anti-E1E2, anti-core and anti-E2 (‘H2’) antibodies. The western blots in FIG. 3 (non-reducing gel) and FIG. 4 (reducing gel) were stained with anti-E1E2 antibody, and show that: E1 expression is not detected in 21-5 cells that do not have the R809 vector; E1 is co-precipitated by the anti-E2 monoclonal antibody, and so E1 and E2 are stably associated as heterodimers; and the anti-core antibody does not precipitate E1 or E2, and so E1/E2 is not associated with core. The western blot in FIG. 5 was stained with anti-core antibody, and shows that core protein is expressed by the 21-5 cells with or without the R809 vector, but it is not precipitated by the anti-core monoclonal antibody. E1/E2 expression could also be detected by ³⁵S-Met labelling.

### Immunofluorescence Stainings and Confocal Microscopy.

Cells were plated on 30 mm coverslips in 24-well plates at a density of 5×10⁴ cells per well. One day after seeding, cells were washed in PBS, fixed in 4% formaldehyde for 30 min and then permeabilized with 0.1% Triton X-100 in PBS for 15 min. Cells were then pretreated with blocking solution (0.5% bovin serum albumin in PBS) for 30 min and incubated for 1 h at room temperature with the primary antibodies diluted in blocking solution according to the manufacturer's instructions. After three washes in PBS, AlexaFluor-conjugated secondary antibodies were added to the cells at a 1:200 dilution for 1 h at room temperature. After washings with PBS, coverslips were washed in distilled water, and mounted in aqueous mounting medium Vectashield with DAPI (4′,6′-diamidino-2-phenylindole).

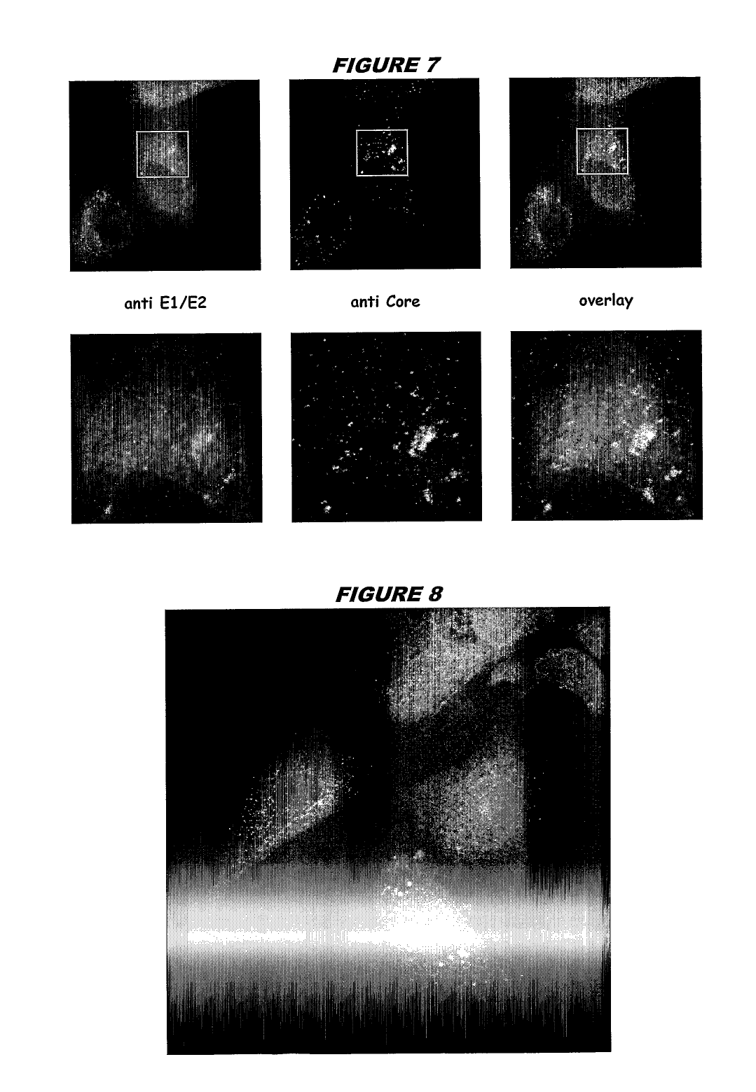

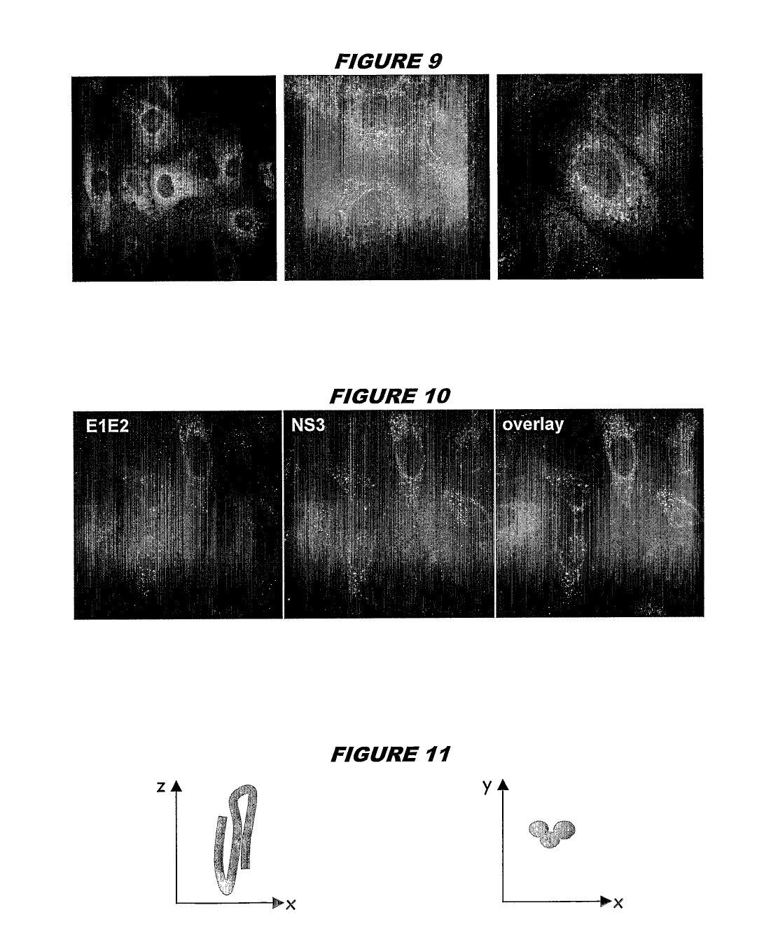

Immunofluorescence analysis of 21-5/R809 cells showed co-localisation of E1 and E2 (dots in 20-30% of cells), and also showed that core protein was accumulating with the E1/E2 complex (FIGS. 6 & 7). The visible fluorescent spots with anti-core had the same shape as those seen with anti-E1/E2. As shown in FIG. 7, in some cases the spots were only partially overlapping, suggesting that the core and E1/E2 proteins were trying to accumulate in the same dot. When different cells were investigated individually, some cells with E1/E2 dots were not expressing core protein, and some with core dots were not expressing E1/E2 (FIG. 8), and so the cells were not homogenous, although those expressing both core and E1/E2 could easily be distinguished and separated from the others. The shape, dimension and intracellular distribution of E1/E2 spots also varied (FIG. 9). This variability could arise from differences in viral quasi-species, differences in transfection efficiency (different clones), or it could be identical cells at different stages in the viral life cycle.

Further immunofluorescence analysis on Huh-7, Huh-7R809, 21-5 and 21-5R809 cells to visualize the intracellular distribution of the HCV glycoproteins was carried out. In the experiment shown in FIG. 21B, we used the anti-E1/E2 chimpanzee antisera L559 [85] to reveal both E1 and E2. However, similar patterns were obtained by using monoclonal anti-E2 antibodies either of human or murine origin. Both the Huh-7R809 and the 21-5R809 showed a large variability in the level of expression of E1/E2 (top panel of FIG. 21B), most likely depending on the fact that they are not of clonal origin. Overall, E1/E2 appeared to have an ER-like distribution, but a significant subset of cells showed a concentration of the two structural proteins in dot-like structures (FIG. 21B) that resemble the ones enriched in NS3 and NS5A described in cells harboring subgenomic replicon [89-92]. Although these structures were present in both cell types, as evidenced by the enlargement of a representative cell, the shape and localization of the structures were not equivalent (bottom of FIG. 21B). As a general observation, in Huh-7 the dot-like structures looked more diffused and to some extent localized at the edge of the cells, whereas in 21-5R809 they appeared closer to the nucleus and more compact.

Co-Localization of E1/E2 with Other HCV Proteins in 21-5R809.

Further experiments showed that E1/E2 also co-localises with NS3 (FIG. 10) and NS5a.

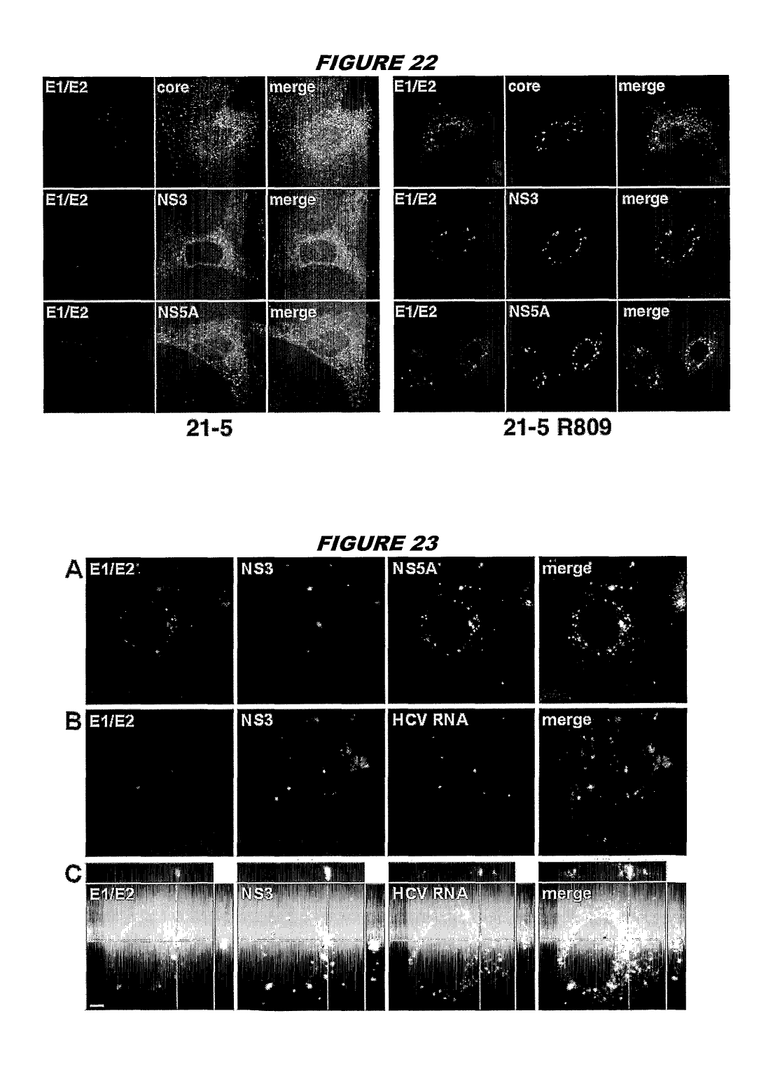

In Huh-7 cells supporting active RNA replication of a subgenomic replicon, both NS3 and NS5A have been shown to localize with newly synthesized HCV RNA on distinct dot-like structures that could represent HCV replicative complexes [90, 91, 93]. Moreover, purified membrane fractions containing HCV nonstructural proteins were found to associate to the endogenous replicon RNA and when supplied with dNTPs, were capable of replicating this RNA in vitro [89-92, 94-97]. We have shown that in 21-5R809 cells, E1/E2 were also organized in dense structures dot-like shaped. Next we sought to determine whether in 21-5 and 21-5R809 cells the nonstructural HCV proteins were also organized in any kind of structure and if there was co-localization with E1/E2. We performed confocal microscopy revealing E1/E2 by L559 antisera in combination with core, NS3 and NS5A respectively. Results are shown in FIG. 22.

In the left panel, the distribution of core (top) NS3 (middle) and NS5A (bottom) was revealed in the 21-5 cells harboring the full length replicon. In these cells core, NS3 and NS5A showed an ER-like distribution with a certain number of areas of dense localization. The core protein appeared to form a significant number of dot-like structures, plus some ring-shaped structures as described in the literature (34). The two HCV nonstructural proteins NS3 and NS5A showed a distribution pattern more ER-like, with small dots mostly localized in the perinuclear region (left panel of FIG. 22, middle and lower rows). Thus, the pattern of NS3 and NS5A appeared dissimilar from the one found in cells harboring subgenomic replicon, in which formation of high number of bigger dots was described [90-93, 98]. The distribution pattern of these proteins appeared different in 21-5R809 cells, in which E1 and E2 were expressed at high level (FIG. 22, right panel). In this context, the core, NS3 and NS5A proteins did form dot-like structures paralleled by a less intense ER-like distribution. Remarkably, dots formed by E1/E2 showed an extensive co-localization with similar structure formed by core, NS3 or NS5A (FIG. 22, right panel). On the other hand, the diffuse ER-like pattern was similar for the four proteins analyzed separately but it did not result in strict co-localization of these species.

Moreover, simultaneous staining for NS3, NS5A and E1/E2 in 21-5R809 cells was achieved using anti-isotype fluorescinate secondary antibodies. Alternatively to NS5A, nascent cytoplasmic RNA was labeled with an anti-BrUTP antibody. Results from these experiments are shown in FIG. 23. The color resulting in the merge picture revealed co-localization of the different species in any combination. Association of the NS3 species with E1/E2, and NS5A or viral RNA was uncovered by the blue color getting lighter. The yellow color was generated by co-localization of E1/E2 and NS5A (or viral RNA), whereas the white areas denoted complete co-localization of all three species. Some of these dot-like structures with triple staining are also shown by z-sections in FIG. 23C (top and right strips).

As shown in FIG. 23A, a broad co-localization of E1/E2 with either NS3 or NS5A was observed. Moreover, several of these dot-like structures were positive for all three species analyzed. These findings suggest a common distribution of the HCV proteins expressed in the context of the replicon that generated structures in which all species co-localized.

An analogous situation was observed for the distribution of the nascent viral RNA in comparison to other viral proteins (FIGS. 23B and 23C). In these experiments, cells were treated with the RNA precursor BrUTP for 2 and 4 hrs in the presence of actinomycin D before fixation for immunofluorescence staining and confocal analysis. As shown in the FIG. 23B, at 2-hour pulse the nascent RNA associated with discrete structures corresponding to the dots in which also E1/E2 and NS3 accumulated. When cells were pulsed with BrUTP for a longer period (4 hours), the labeled viral RNA could be found not only in the dots together with E1/E2 and NS3 but also spread throughout the cells (FIG. 23C). This is consistent with the double function of the HCV RNA that constitutes the virus genome that has to be replicated but also functions as messenger. Co-localization of nonstructural proteins with nascent RNA has been reported to represent viral replicative complexes [89-92]. Our data suggest that the two HCV glycoproteins, when expressed, also associate with these complexes.

Confocal microscopy of the immunofluorescence dots revealed that E1/E2 label was distributed along cells with a maximum length of about 3 μm, suggesting a tubular structure (FIG. 11).

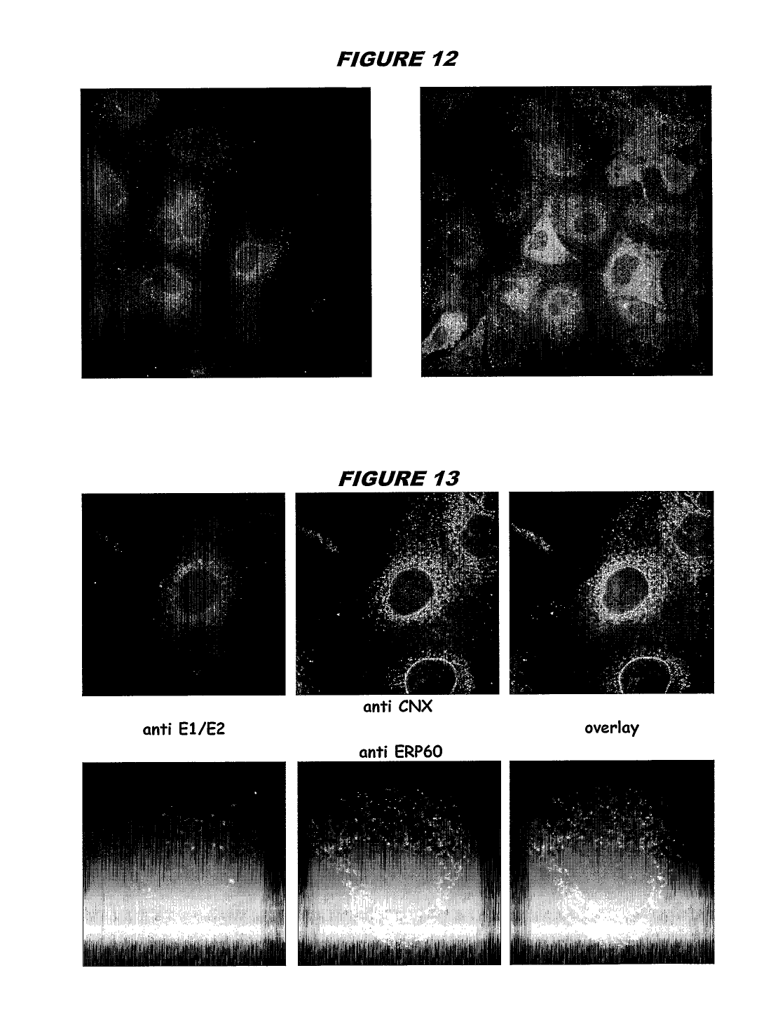

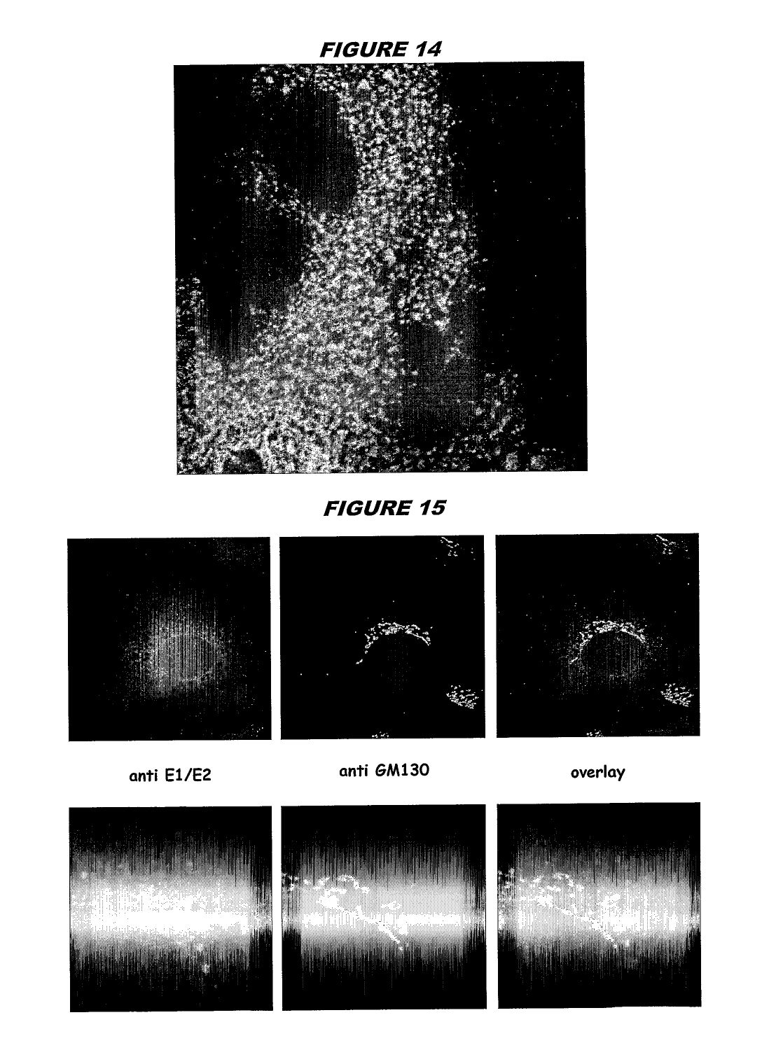

Huh7 cells transfected with the R809 vector (i.e. no HCV presence) also showed the E1/E2 dots, but the labelling was around 5-fold lower than in the 21-5 cells (50% vs. 10%) and the intracellular distribution seemed slightly different (FIG. 12). This suggests that E1 and E2 expressed from the R809 vector can assemble in the cells, but that they cannot proceed further along viral self-assembly without the core (and other) proteins.

### Intracellular Localization of E1/E2

In most reports, the HCV proteins have been described to associate to the ER or ER-derived membrane, with some work pointing to the involvement of the Golgi apparatus or of the trans-Golgi network [89, 90, 92, 98-100]. We sought to catch the location of E1/E2 in 21-5R809 cells by confocal analysis via simultaneous labeling of the two HCV glycoproteins with known markers of different cellular compartments.

Briefly, to assess intracellular localisation, cells were stained also with anti-calnexin (CNX) or anti-ERP60, both of which are markers for the endoplasmic reticulum (membrane and lumen, respectively). E1/E2 did not totally co-localise with CNX, and some E1/E2 was seen in the periphery of the cell, suggesting that the protein complex had progressed beyond the ER (FIGS. 13 & 14). Cells were then labelled with anti-GM130, a marker for the cis-Golgi. Co-localisation of E1/E2 and GM130 was not seen (FIG. 15). E1/E2 were sensitive to endoglycosidase H treatment, indicating that they are ER-retained.

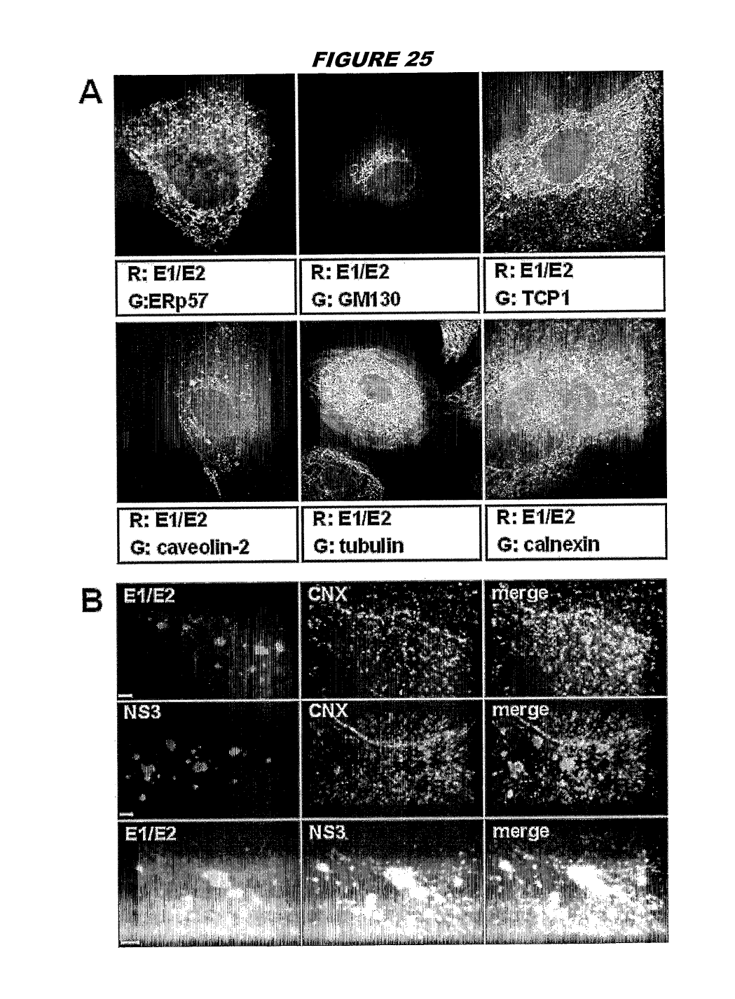

In more detail, the results of such analysis, shown in FIG. 25A, allowed us to conclude that E1/E2 did not co-localize with the Golgi apparatus (GM-130) or mitochondria (TCP1) and that they were not attached to microtubules (α-tubulin) or associated to caveolin-2. A broad co-localization with the two ER markers, the soluble lumenal protein disulfide isomerase Erp57 and the membrane-spanning protein calnexin, seemed also unlikely, as shown in FIG. 25A, where just very limited areas of possible merge are visible. E1/E2, Erp57 and calnexin showed a broad reticular pattern characterized by the absence of significant overlie. On the other hand, the distribution of both species around the areas surrounding the dot-like structures formed by E1/E2 was much more difficult to judge.

Since calnexin has been often associated to the HCV glycoproteins higher magnification confocal scanning analysis on several distinct samples was performed. FIG. 25B shows representative images of such analyses. Co-localization of calnexin with E1/E2 was limited to few minor areas at the edges of the dots (FIG. 25B, top row). More clearly, the NS3 species lied completely separated from the calnexin (FIG. 25B, middle row) while strong co-localization was unambiguous for E1/E2 with NS3 (FIG. 25B, bottom row). In the latter, the two species showed a dense core of co-localization and few peripheral areas of single species distribution.

In conclusion, the ER membrane spanning chaperone calnexin appeared essentially co-distributed with the E1/E2 but co-localized only in a very minor fractions. Furthermore, calnexin also appeared to be excluded from the central areas of the dots. Thus, in a high percentage of 21-5R809 cells, core, E1/E2, NS3 and NS5A HCV proteins accumulated and strictly co-localized in dense structures around which the only recognizable cellular marker is an ER transmembrane chaperone. Taken together these data were consistent with a localization of the HCV proteins in an ER-derived compartment.

### Cellular Distribution of NS3 and NS5A in the Presence or Absence of E1/E2 Expression.

Association of HCV nonstructural proteins in dot-like structures, particularly NS3 and NS5A, has already been reported in cells harboring subgenomic replicons.

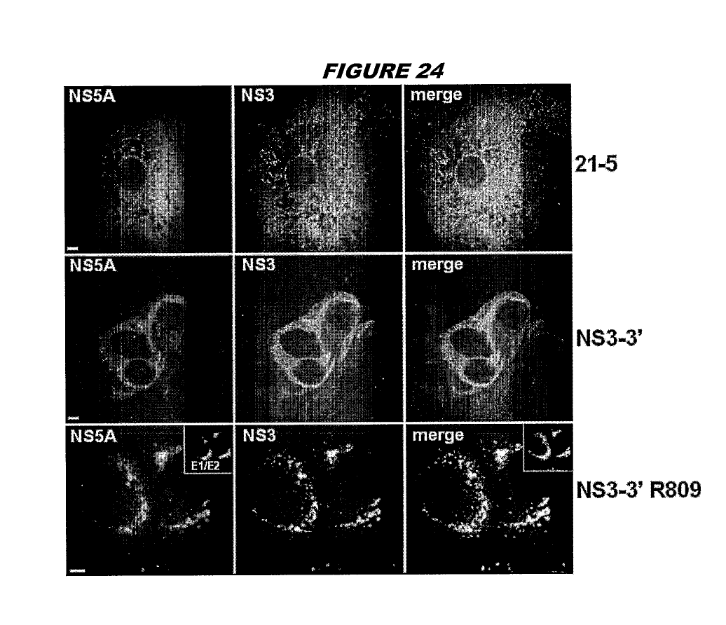

To unravel the relative contribution of the different viral proteins to the process of dot formation, we analyzed the distribution of the nonstructural proteins NS3 and NS5A in 21-5 cells, a system in which there is detectable expression of the core protein but not of the two glycoproteins, and in Huh-7 harboring subgenomic replicon, either expressing or not expressing E1 and E2. The Huh-7 cell line supporting replication of the subgenomic replicon, corresponding to the NS3-3′ HCV-1b region, was described in reference 1. We transduced this cell line by the lentiviral vector carrying E1E2p7 gene from genotype 1a as shown in FIG. 21A. The expression of the two glycoproteins was checked by Western blot, whereas immunofluorescence analysis stated that virtually 100% of the cells expressed E1/E2. Representative images from confocal analysis on these cell types are shown in FIG. 24. In 21-5 cells, both NS3 and NS5A showed a diffuse ER-like distribution with some dot-like areas of reduced size all over the cell surface (FIG. 24, top panel). Co-localization of the two nonstructural proteins appeared to be confined to few visible dots while the reticular-distributed proteins did not seem to associate. In NS3-3′ cells, the ER-like pattern was reduced and paralleled by an increased number of larger dots containing both proteins (FIG. 24, middle row). The simultaneous presence of NS3 and NS5A in dot-like structures was much more defined in NS3-3′R809 cells (FIG. 24, bottom row). In these cells, these structures increased in both number and size, meanwhile a simultaneous reduced ER-like distribution of both species was observed. Once again, co-localization of the two nonstructural proteins was preferentially restricted to the dot areas. The simultaneous labeling of E1/E2 in these cells is shown in the insets (FIG. 24, bottom row). The result clearly showed extensive co-localization of the two nonstructural proteins with E1/E2 in the dots plus several areas in which two species co-localized. These observations were consistent with a synergistic effect of E1/E2 expression on induction of dot-like structures formation in cells harboring both full-length or subgenomic replicons.

Taken together these observations indicated a complex pattern of interaction that could drive the formation of the dot-like structures in these cells. All the viral proteins analyzed so far showed either a reticular distribution or concentrated in dot-like structures, whose numbers and locations appeared to depend not on the presence of a particular viral protein but on the context in which it was expressed. Furthermore, it was also clear that a subtype of cells allowing HCV RNA replication accumulates all available viral products in huge structures that could correspond to pre-budding areas.

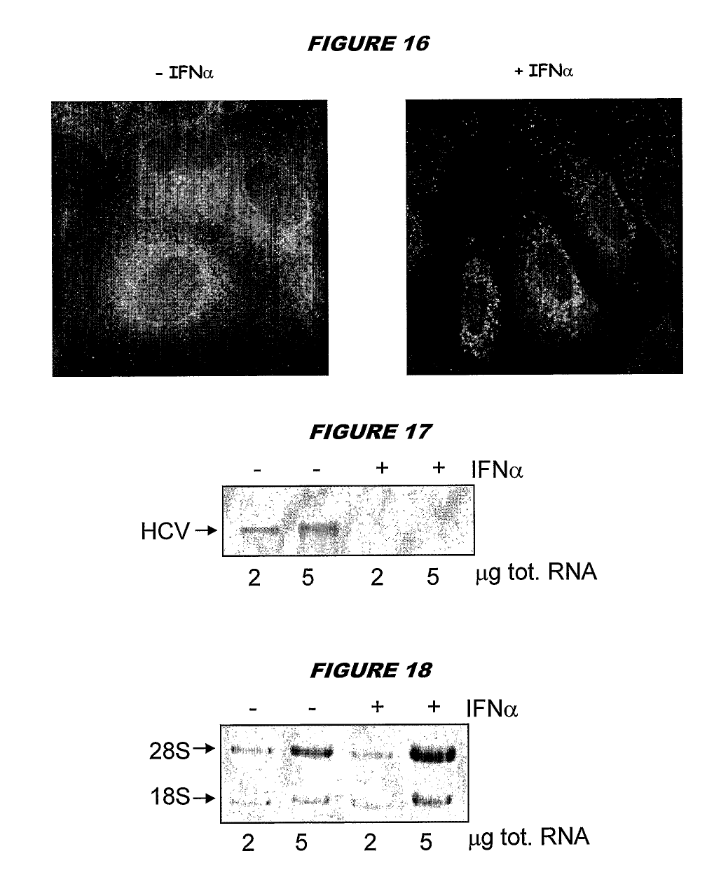

Interferon alpha (IFN-α) is known to inhibit HCV replication in cell culture [101]. Incubation of the 21-5/R809 cells with IFN-α changed the ER-pattern distribution of E1 and E2 (FIG. 16), and caused a decrease in HCV RNA levels both absolutely (FIG. 17) and relative to ribosomal RNA (FIG. 18).

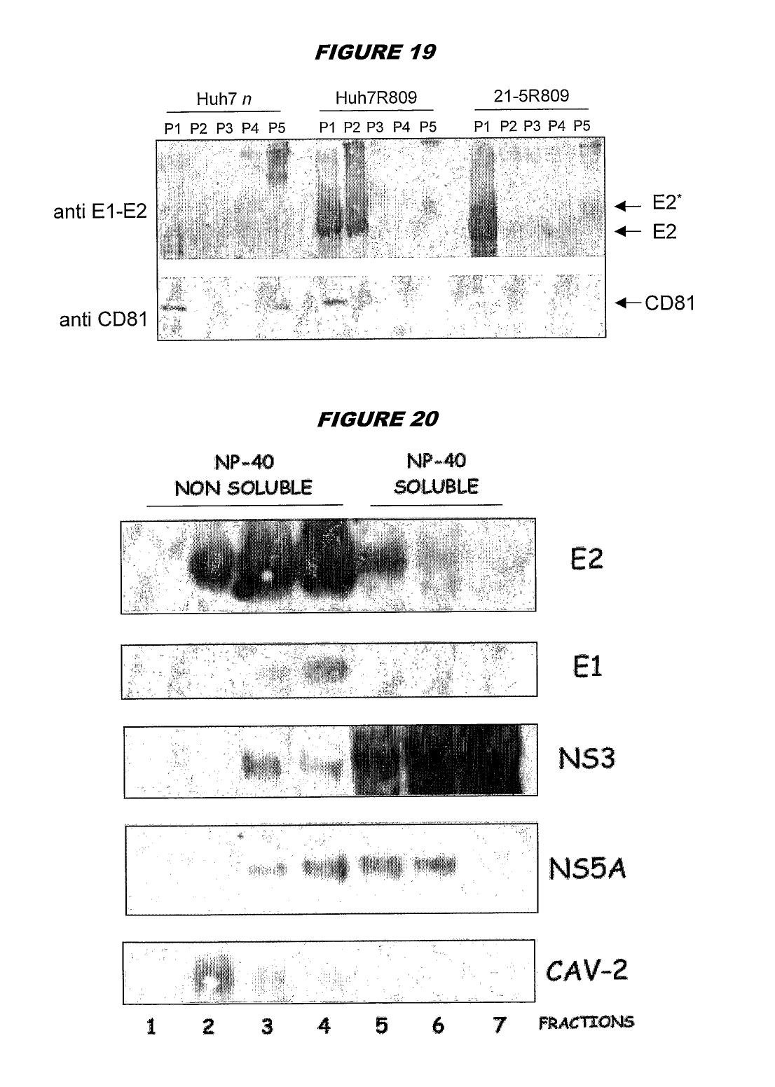

Differential centrifugation was used in order to purify exosomes from (a) Huh7 cells; (b) R809-transfected Huh7 cells; and (c) 21-5/R809 cells. As shown in FIG. 19, the P5 fraction from one centrifugation experiment detected a faint band which would match a Golgi-modified E2 protein. CD81 could also be detected.

### Labeling of De Novo-Synthesized Viral RNA.

Cells were plated on coverslips in 24-well plates at a density of 5×10⁴ per well. One day after seeding, cells were transfected with Bromo-UTP (BrUTP)), using lipofectamine 2000 according to the manufacturer's instructions. Briefly, 50 μl of 12 mM BrUTP in Optimem were added to 1 μl of lipofectamine 2000 diluted in 50 μl of Optimem. After 20 min incubation at room temperature, the BrUTP-lipofectamine complex was added directly to the cells in 500 μl of DMEM complete medium in the presence of actinomycin D (5 μg/μl). After 2 or 4 h of incubation, cells were fixed in 4% paraformaldehyde in PBS and permeabilized in 1% Triton X-100 in PBS. HCV proteins were identified as described above, while newly synthesized viral RNA was detected using a mouse anti-BrUTP antibody at 2 μg/ml. Primary antibodies were detected with AlexaFluor-conjugated secondary antibodies diluted 1:200 in PBS/BSA 0.5%. By labeling de novo-synthesized HCV RNA, we showed that the complexes of the invention constitute a site of viral RNA synthesis.

### Membrane Floatation Assay.

To better characterize the nature of the intracellular membranes that associate with the HCV proteins, we performed subcellular fractionation studies to separate membrane and cytosolic fractions.

Cells were lysed in 1 ml of hypotonic buffer (10 mM Tris-Hcl pH 7.5, 10 mM KCl, 5 mM MgCl₂) and homogenized with 50 strokes in a Wheaton loose-fitting dounce homogenizer. Nuclei and unbroken cells were removed by centrifugation at 1000×g for 5 min at 4° C. To characterize the membrane structures, postnuclear supernatants of cell lysates were treated with 1% Triton X-100 at 4° C. or 37° C. for 30 min. Cell lysates were then brought to 55% sucrose by mixing with sucrose 80% in low salt buffer (50 mM Tris-HCl pH 7.5, 25 mM KCl, 5 mM MgCl₂) and overlaid with 6 ml of 35% sucrose and finally with 3 ml of 5% sucrose. The gradient was centrifugated at 38000 rpm in a Beckman SW40 rotor for 20 hours at 4° C. After centrifugation, 1 ml fractions were collected from the top of the gradient, diluted in PBS and centrifuged for 60 min at 350000×g to precipitate the material. The pellets were resuspended in SDS sample buffer, separated on 10% polyacrylamide gels and transferred to nitrocellulose membranes. After blocking, the membrane was incubated with the primary antibody for 1 hour at room temperature, followed by the appropriate species-specific horseradish peroxidase conjugate secondary antibody, for an additional 1 hour at room temperature.

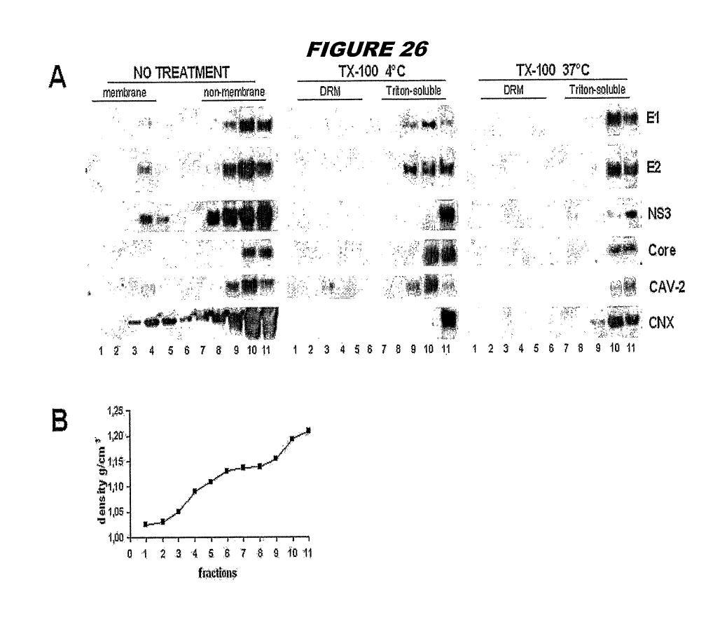

Cellular extracts from the 21-5R809 cell line were fractionated on sucrose gradients and examined for the presence of the structural and nonstructural HCV proteins, the ER membrane-associated protein calnexin, and the cytoplasmic lipid raft marker caveolin-2. Membrane-associated proteins were expected to float to an equilibrium density within the gradient. Indeed, a good proportion of both E1/E2 and NS3 were found in the membrane fraction (fractions 4-5) while the core protein could just be detected in the soluble fraction, most likely due to its lower level of expression (FIG. 26A, left panel). The ER marker calnexin was distributed in both the membrane and the cytosolic fractions, as commonly found using these biochemical procedures. When the cell extract was treated prior to fractionation with 1% Triton X-100 at 4° C.—a condition that release the ER proteins to the cytosol-, all HCV proteins were found in the cytosolic fractions at the bottom of the gradient (FIG. 26A, middle panel). Therefore, in agreement with a previous report [95], Triton X-100 treatment of the cell extract resulted in the dissociation of the HCV replication complex with the ER membranes. The same treatment did not disrupt the detergent resistant membranes (DRMs) that co-fractionate with Cav-2 (FIG. 26A, middle panel). Incubation with TX-100 at 37° C. led to the complete solubilization of the DRM proteins (FIG. 26A, left panel). Taken together these data indicated that the HCV replication/assembly complex described so far associates with membrane structures of ER origin.

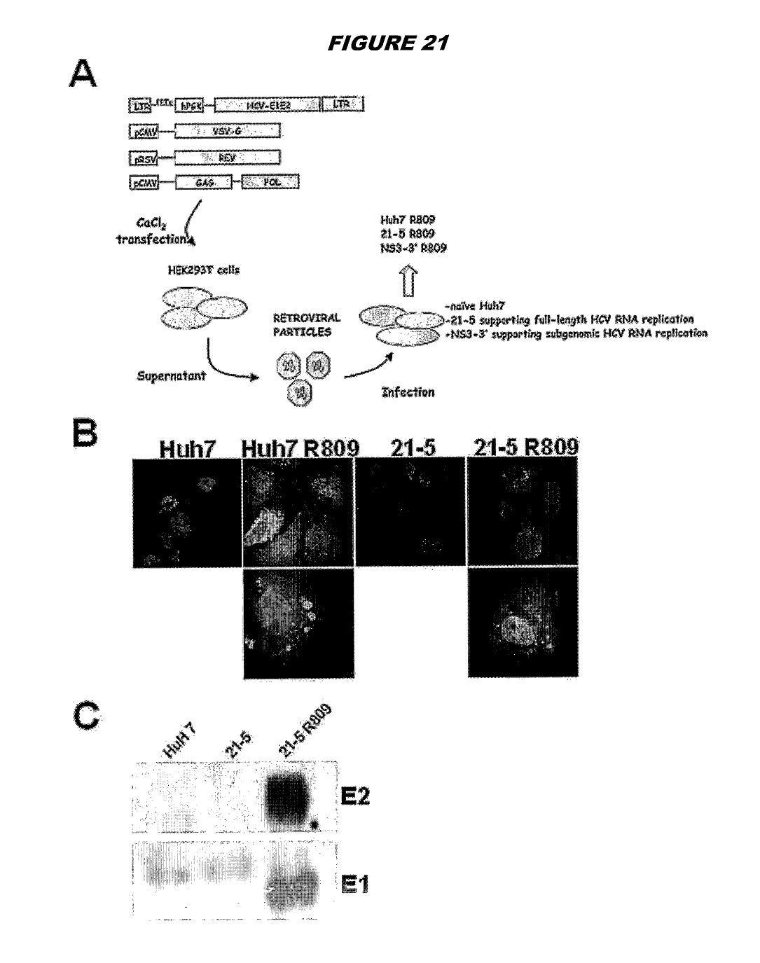

Membrane flotation analysis demonstrated that the majority of E2 was associated with membranes that are resistant to treatment with 1% NP40 and co-fractionate with caveolin-2 (FIG. 20).

### SUMMARY

In this report we describe a new cell system that could be helpful for the analysis of the subcellular localization of the HCV structural and nonstructural proteins and for studying the virus budding mechanism. We have established a stable and detectable expression of E1E2 (and p7) in a cell line harboring a full length genotype 1b replicon and characterized the localization of the two glycoproteins in relation with the nonstructural ones, viral RNA and intracellular markers. In this system, the expression of the two glycoproteins remained reasonably persistent and, as expected, resulted in the formation of E1E2 heterodimers. Moreover, the chromosomal expression of the two glycoproteins prevents the genetic drift of the two species, thus avoiding the accumulation of mutations that could influence E1E2 stability. Two patterns of protein distribution were observed: one “reticular”, similar to any other ER marker labeling, and a “patched” one, in which localized accumulation of viral proteins generated dot-like structures. In their reticular distribution, the HCV proteins did not preferentially co-localize with each other or with the standard markers used to identify subcellular districts.

All HCV proteins interact with host cell membranes either directly as membrane-binding proteins, or indirectly, inducing specific patterns of alterations. We confirmed that the isolate expression of E1E2p7 was sufficient to induce dot-like structures in naïve Huh7 cells (FIG. 21B). However, the number, the shape and the localization of these structures were depending on the context of co-expressed viral proteins. In fact, we demonstrated that the reinforced expression of E1E2 in cells harboring the full length or the subgenomic replicon induced the reduction of NS3 and NS5A reticular distribution and their concomitant accumulation in more defined areas where all the viral components concentrated (FIGS. 22 and 24). Conversely, the exogenous expressed E1/E2 are organized in sharper, larger and more perinuclear dot-like structures when expressed in the context of the replicon when compared with their cellular localization in naïve Huh7 cells.

It will be understood that the invention has been described by way of example only and modifications may be made whilst remaining within the scope and spirit of the invention.

### REFERENCES

### The Contents of Which are Hereby Incorporated by Reference

[1] Lohmann et al. (1999) Science 285:110-113.
[2] Blight et al. (2000) Science 290:1972-1974.
[3] WO01/89364.
[4] Pietschmann et al. (2002) J Virol 76:4008-21.
[5] Klein et al. (2004) J Virol 78:9257-69.
[6] Wakita et al. (2005) Nature Medicine 11:791-6. Erratum in Nature Med 11:905.
[7] Lindenbach et al. (2005) Science 309:623-6.
[8] Zhong et al. (2005) PNAS USA 102:9294-9.
[9] Nakabayashi et al. (1982) Cancer Res 42:3858-63.
[10] WO2004/044182
[11] http://hcv.lanl.gov/content/hcv-db/classification/genotable.html
[12] Simmonds et al. (1996) J Gen Virol 77:3013-24.
[13] Gennaro (2000) Remington: The Science and Practice of Pharmacy. 20th edition, ISBN: 0683306472.
[14] WO03/009869.
[15] Almeida & Alpar (1996) J Drug Targeting 3:455-467.
[16] Agarwal & Mishra (1999) Indian J Exp Biol 37:6-16.
[17] Vaccine Design . . . (1995) eds. Powell & Newman. ISBN: 030644867X. Plenum.
[18] WO00/23105.
[19] WO90/14837.
[20] U.S. Pat. No. 5,057,540.
[21] WO96/33739.
[22] EP-A-0109942.
[23] WO96/11711.
[24] WO00/07621.
[25] Barr et al. (1998) Advanced Drug Delivery Reviews 32:247-271.
[26] Sjolanderet et al. (1998) Advanced Drug Delivery Reviews 32:321-338.
[27] Niikura et al. (2002) Virology 293:273-280.
[28] Lenz et al. (2001) J Immunol 166:5346-5355.
[29] Pinto et al. (2003) J Infect Dis 188:327-338.
[30] Gerber et al. (2001) Virol 75:4752-4760.
[31] WO03/024480
[32] WO03/024481
[33] Gluck et al. (2002) Vaccine 20:B10-B16.
[34] EP-A-0689454.
[35] Johnson et al. (1999) Bioorg Med Chem Lett 9:2273-2278.
[36] Evans et al. (2003) Expert Rev Vaccines 2:219-229.
[37] Meraldi et al. (2003) Vaccine 21:2485-2491.
[38] Pajak et al. (2003) Vaccine 21:836-842.
[39] Kandimalla et al. (2003) Nucleic Acids Research 31:2393-2400.
[40] WO02/26757.
[41] WO99/62923.
[42] Krieg (2003) Nature Medicine 9:831-835.
[43] McCluskie et al. (2002) FEMS Immunology and Medical Microbiology 32:179-185.
[44] WO98/40100.
[45] U.S. Pat. No. 6,207,646.
[46] U.S. Pat. No. 6,239,116.
[47] U.S. Pat. No. 6,429,199.
[48] Kandimalla et al. (2003) Biochemical Society Transactions 31 (part 3):654-658.
[49] Blackwell et al. (2003) J Immunol 170:4061-4068.
[50] Krieg (2002) Trends Immunol 23:64-65.
[51] WO01/95935.
[52] Kandimalla et al. (2003) BBRC 306:948-953.
[53] Bhagat et al. (2003) BBRC 300:853-861.
[54] WO03/035836.
[55] WO95/17211.
[56] WO98/42375.
[57] Beignon et al. (2002) Infect Immun 70:3012-3019.
[58] Pizza et al. (2001) Vaccine 19:2534-2541.
[59] Pizza et al. (2000) Int J Med Microbiol 290:455-461.
[60] Scharton-Kersten et al. (2000) Infect Immun 68:5306-5313.
[61] Ryan et al. (1999) Infect Immun 67:6270-6280.
[62] Partidos et al. (1999) Immunol Lett 67:209-216.
[63] Peppoloni et al. (2003) Expert Rev Vaccines 2:285-293.
[64] Pine et al. (2002) J Control Release 85:263-270.
[65] Domenighini et al. (1995) Mol Microbiol 15:1165-1167.
[66] WO99/40936.
[67] WO99/44636.
[68] Singh et al] (2001) J Cont Release 70:267-276.
[69] WO99/27960.
[70] U.S. Pat. No. 6,090,406
[71] U.S. Pat. No. 5,916,588
[72] EP-A-0626169.
[73] WO99/52549.
[74] WO01/21207.
[75] WO01/21152.
[76] Andrianov et al. (1998) Biomaterials 19:109-115.
[77] Payne et al. (1998) Adv Drug Delivery Review 31:185-196.
[78] Stanley (2002) Clin Exp Dermatol 27:571-577.
[79] Jones (2003) Curr Opin Investig Drugs 4:214-218.
[80] WO99/11241.
[81] WO94/00153.
[82] WO98/57659.
[83] European patent applications 0835318, 0735898 and 0761231.
[84] Knipe & Howley Fields Virology (4th edition, 2001). ISBN 0-7817-1832-5.
[85] Choo, Q. L., G. Kuo, et al. (1994) Proc Natl Acad Sci USA 91:1294-8.
[86] Follenzi, A., L. E. et al., (2000) Nat Genet. 25:217-22.
[87] Dull, T., R. et al., (1998) J Virol 72:8463-71.
[88] Cavalieri, S., S. et al., (2003) Blood 102:497-505.
[89] Aizaki, H., K. J. et al., (2004) Virology 324:450-61.
[90] Gosert, R., D et al., (2003) J Virol 77:5487-92.
[91] Moradpour, D., et al., (2004) J Virol 78:7400-9.
[92] Shi, S. T., K. J. et al., (2003) J Virol 77:4160-8.
[93] Shi, S. T., S. J. (2002) Virology 292:198-210.
[94] Ali, N., K. D. (2002) J Virol 76:12001-7.
[95] El-Hage, N., and G. Luo. (2003) J Gen Virol 84:2761-9.
[96] Hardy, R. W., J. et al., (2003) J Virol 77:2029-37.
[97] Lai, V. C., S. (2003) J Virol 77:2295-300.
[98] Mottola, G., G. et al., (2002) Virology 293:31-43.
[99] Hijikata, M., H. et al., (1993) Proc Natl Acad Sci USA 90:10773-7.
[100] Konan, K. V., T. H. et al., (2003) J Virol 77:7843-55.
[101] Chung et al. (2001) PNAS USA 98:9847-9852.

1. A cell comprising, (a) a hepatitis C virus genome, and/or nucleic acid encoding a hepatitis C virus genome; and (b) nucleic acid encoding one or both of hepatitis C virus proteins E1 and E2.

2. A cell according to claim 1, wherein the hepatitis C virus is replicating and hepatitis C virus protein(s) E1 and/or E2 is/are expressed in addition to any E1 and/or E2 protein that is expressed as a result of the hepatitis C virus life cycle.

3. A cell according to claim 1, wherein the E1 and/or E2 is/are expressed in a form separate from the E1/E2 that arises from proteolytic processing of HCV polyprotein expressed from the HCV genome during the viral life cycle.

4. A cell according to claim 1, wherein the cell (a) includes a hepatitis C virus genome, and/or nucleic acid encoding a hepatitis C virus genome; (b) expresses hepatitis C virus proteins E1 and E2; and (c) can grow in an in vitro culture.

5. A cell according to claim 1, wherein the cell contains the following two nucleic acids in trans: (a) a hepatitis C virus genome; and (b) a nucleic acid encoding hepatitis C virus protein E1 and/or E2.

6. A cell according to claim 1, wherein the cell contains the following two nucleic acids in trans: (a) nucleic acid encoding a hepatitis C virus genome; and (b) a nucleic acid encoding hepatitis C virus protein E1 and/or E2.

7. A method for preparing a cell according to claim 1, comprising the steps of (a) introducing into the cell a hepatitis C virus genome and/or nucleic acid encoding a hepatitis C virus genome; and (b) introducing into the cell nucleic acid encoding one or both of hepatitis C virus proteins E1 and E2.

8. The method according to claim 7, wherein steps (a) and (b) are performed separately.

9. The method according to claim 7, wherein steps (a) and (b) are performed simultaneously.

10. An in vitro method for culturing hepatitis C virus in a cell comprising preparing a cell according to claim 7 followed by (c) culturing the resulting cell.

11. A method for preparing hepatitis C virus E1 and E2 proteins, comprising the steps of: (a) culturing a cell of claim 1; and (b) purifying the E1 and E2 proteins from the cultured cells.

12. The method of claim 11, wherein the E1 and E2 proteins are co-purified with one or more of the hepatitis C virus non-structural proteins.

13. The method of claim 11, wherein the E1 and E2 proteins are in the form of a complex.

14. An E1/E2 complex obtainable by the method of claim 13.

15. The E1/E2 complex of claim 14, wherein the complex is a virion like particle.

16. The E1/E2 complex of claim 15, wherein the virion like particle comprises one or more of the hepatitis C virus non-structural proteins and/or RNA.

17. A vaccine for the treatment or prevention of hepatitis C virus comprising the E1/E2 complex of claim 13.

18. A cell including a hepatitis C virus genome, and/or nucleic acid encoding a hepatitis C virus genome, into which nucleic acid encoding one or both of hepatitis C virus proteins E1 and E2 can be introduced.

19. A cell including nucleic acid encoding one or both of hepatitis C virus proteins E1 and E2, into which a hepatitis C virus genome and/or nucleic acid encoding a hepatitis C virus genome can be introduced.
